(19) United States
(12) Patent Application Publication
(10) Pub. No.: US 2021/0272672 A1
(43) Pub. Date: Sep. 2, 2021

(54) PERSONALIZED PAIN MANAGEMENT METHOD, DEVICE AND COMPUTER PROGRAM

(71) Applicant: Ju Eun SUNG, Seoul (KR)

(72) Inventor: Ju Eun SUNG, Seoul (KR)

(21) Appl. No.: 17/318,673

(22) Filed: May 12, 2021

Related U.S. Application Data
(63) Continuation of application No. PCT/KR2019/015277, filed on Nov. 11, 2019.

(30) Foreign Application Priority Data
Nov. 16, 2018 (KR) 10-2018-0141472

(51) Int. Cl. G16H 20/30; G16H 10/20; G16H 50/70; G16H 10/60; G16H 15/00; G16H 50/50; G16H 40/67; G16H 40/20; G16H 20/10; G01C 22/00; G01P 13/00; G01K 13/20; G06F 3/0482; G06F 3/0481; G06F 3/0484; A61B 5/024; A61B 5/0205; A61B 5/021; A61B 5/11; A61B 5/00

(52) U.S. Cl. CPC G16H 20/30; A61B 2562/0219; G16H 50/70; G16H 10/60; G16H 15/00; G16H 50/50; G16H 40/67; G16H 40/20; G16H 20/10; G01C 22/006; G01P 13/00; G01K 13/20; G06F 3/0482; G06F 3/04817; G06F 3/04815; G06F 3/04845; A61B 5/024; A61B 5/02055; A61B 5/021; A61B 5/1118; A61B 5/4266; A61B 5/4824; A61B 2562/029; G16H 10/20

(57) ABSTRACT

The present disclosure relates to a personalized pain management method, device, and computer program, and to a method, device and computer program capable of managing pain of a user by setting a baseline for a period of time or a number of times to accept pain for each activity of a user, and using the intensity, occurrence part and duration of pain within the baseline.

### CROSS-REFERENCE TO RELATED APPLICATION

The present application is a continuation of International Patent Application No. PCT/KR2019/015277, filed on Nov. 11, 2019, which is based upon and claims the benefit of priority to Korean Patent Application No. 10-2018-0141472 filed on Nov. 16, 2018. The disclosures of the above-listed applications are hereby incorporated by reference herein in their entirety.

### TECHNICAL FIELD

The present disclosure relates to a method and device for customized pain management according to a pain state of a user suffering from chronic pain, and more specifically, to a method, device, and computer program for managing pain information of a user based on a baseline set for each activity.

### BACKGROUND ART

As demand for mobile devices such as smartphones, smartwatches, and tablets increases rapidly, studies of user interface methods related to touchscreens provided in smartphones and tablets are being actively conducted. In particular, studies are being conducted so that a smartphone or tablet can provide an interface method that is close to intuition, related to user experience, and accordingly, interface methods that correspond to various types of user intuition are being disclosed.

In addition, the development of applications that can be used in smartphones or tablets is increasing rapidly, and in particular, the development of healthcare applications capable of managing the health of users is actively progressing. Conventional healthcare applications adopt a format for managing health information after receiving the health information of users. However, the user simply inputs his or her health information and has no choice but to check the progress of the input health information, thus making it difficult to actively participate. Accordingly, the need for healthcare applications in which users can more actively participate in health management has increased.

In particular, in the case of users suffering from chronic pain, careful management of environmental information related to atmospheric pressure, temperature, and humidity, consumption or non-consumption of medicine, and the intensity and duration of pain for each activity is necessary. Therefore, the needs of users to be able to care for chronic pain in different situations are gradually increasing.

### DISCLOSURE OF INVENTION

### Technical Problem

Various aspects of the present disclosure are directed to providing a method, device and computer program capable of managing pain of a user by setting a baseline for a period of time or a number of times to accept pain for each activity of a user, and using the intensity, occurrence part and duration of pain within the baseline.

The aspects of the present disclosure are not limited to those mentioned above, and other aspects not mentioned will be clearly understood by those skilled in the art from the following description.

### Solution to Problem

A personalized pain management method according to an embodiment of the present disclosure is a method performed by a personalized pain management device, and may include: receiving user input selecting any one activity included in an activity list; setting a baseline indicating a threshold point to which a user is able to endure pain while performing the activity selected by the user by using one or more question prompts or user setup request messages; obtaining pain information of the user occurring while performing the activity whenever an activity based on the set baseline is completed; and setting a new baseline with respect to the activity based on the pain information of the user, wherein the user setup request message is a message requesting that the user directly input a longest time or maximum number of times the user is able to perform the activity without pain; the setting of the baseline includes displaying a question prompt as to whether the activity may be performed without pain for a reference period of time or reference number of times; when a response input is received from the user indicating that the activity may be performed without pain for the reference period of time or reference number of times, displaying a question prompt as to whether the activity may be performed without pain for a period of time or a number of times extended from the reference period of time or reference number of times; when a response input is received from the user indicating that the activity may not be performed without pain within the reference period of time or reference number of times, displaying a question prompt as to whether the activity may be performed without pain for a period of time or a number of times shorter than the reference period of time or reference number of times; and setting a maximum period of time or maximum number of times for the user to endure pain as a baseline.

A personalized pain management device according to an embodiment of the present disclosure includes a user interface capable of receiving user input, a processor, and a memory storing instructions executable by the processor. By executing the instructions, the processor may receive user input selecting any one activity included in an activity list, set a baseline indicating a threshold point to which the user is able to endure pain while performing the activity selected by the user by using one or more question prompts or user setup request messages, obtain pain information of the user occurring while performing the activity whenever an activity based on the set baseline is completed, and set a new baseline with respect to the activity based on the pain information of the user. The setting of the baseline may display a question prompt as to whether the activity may be performed without pain for a reference period of time or reference number of times; when a response input is received from the user indicating that the activity may be performed without pain for the reference period of time or reference number of times, display a question prompt as to whether the activity may be performed without pain for a reference period of time or a number of times extended from the reference period of time or reference number of times; when a response input is received from the user indicating that the activity may not be performed without pain within the reference period of time or reference number of times, display a question prompt as to whether the activity may be performed without pain for a period of time or a number of times shorter than the reference period of time or reference number of times; and set a maximum period of time or maximum number of times for the user to endure pain as a baseline.

In a computer program stored in a recording medium in order to execute a personalized pain management method in combination with a computer as hardware according to an embodiment of the present disclosure, the computer program may include: receiving user input selecting any one activity included in an activity list; setting a baseline indicating a threshold point to which a user is able to endure pain while performing the activity selected by the user by using one or more question prompts or user setup request messages; obtaining pain information of the user occurring while performing the activity whenever an activity based on the set baseline is completed; and setting a new baseline with respect to the activity based on the pain information of the user, wherein the user setup request message is a message requesting that the user directly input the longest time or maximum number of times the user may perform an activity without pain. The setting of the baseline includes displaying a question prompt as to whether the activity may be performed without pain for a reference period of time or reference number of times; when a response input is received from the user indicating that the activity may be performed without pain for the reference period of time or reference number of times, displaying a question prompt as to whether the activity may be performed without pain for a period of time or a number of times extended from the reference period of time or reference number of times; when a response input is received from the user indicating that the activity may not be performed without pain within the reference period of time or reference number of times, displaying a question prompt as to whether the activity may be performed without pain for a period of time or a number of times shorter than the reference period of time or reference number of times; and setting a maximum period of time or maximum number of times for the user to endure pain as a baseline.

### Advantageous Effects of Invention

According to an embodiment of the present disclosure, by setting an accurate baseline for each activity of a user based on pain information of a user, it is possible to efficiently manage the pain of a user for various activities.

In addition, according to an embodiment of the present disclosure, by providing information for managing pain by combining pain information of a user and surrounding environment information such as temperature and humidity, the user is able to manage pain in consideration of real-time surrounding environment information.

In addition, according to an embodiment of the present disclosure, it is possible to provide a customized pain management effect as the pain of a user is relieved or worsened by resetting the baseline according to a predetermined period of the user.

In addition, according to an embodiment the present disclosure, by providing a graph of pain information according to the activity of a user, it is possible to systematically provide pain information about a period, an activity field, an activity amount, an activity time, and a pain range.

In addition, according to an embodiment of the present disclosure, by setting the baseline according to the number of exercises designated by the user, pain according to a specific exercise or activity may be specifically managed. The user may check whether pain occurs due to the change in the number of exercises set by the user, so that special care can be received. In addition, there is an advantage of feeling a sense of achievement as the number of times increases.

The effects of the present disclosure are not limited to those mentioned above, and other effects not mentioned will be clearly understood by those skilled in the art from the following description.

### BRIEF DESCRIPTION OF DRAWINGS

FIG. 1 is a diagram schematically illustrating an operation of a device for providing a personalized pain management method according to an embodiment of the present disclosure.

FIG. 2 is a flowchart illustrating a personalized pain management method according to an embodiment of the present disclosure.

FIGS. 3A and 3B are diagrams illustrating a user interface screen for selecting an activity list according to an embodiment of the present disclosure.

FIGS. 4A to 4F are diagrams illustrating a method of setting a baseline according to an embodiment of the present disclosure.

FIG. 5 is a diagram illustrating a method of directly setting a baseline by a user according to an embodiment of the present disclosure.

FIGS. 6A and 6B are diagrams illustrating a method of resetting a baseline according to an embodiment of the present disclosure.

FIGS. 7A to 7C are diagrams illustrating a method of recording an activity of a user according to a baseline according to an embodiment of the present disclosure.

FIGS. 8A to 8D are diagrams illustrating pain information according to activities of a user according to an embodiment of the present disclosure.

FIGS. 9A and 9B are diagrams illustrating a graph by year in which pain information according to activities of a user is accumulated according to an embodiment of the present disclosure.

FIGS. 10A to 10D are diagrams illustrating a user interface through which information on pain may be input according to an embodiment of the present disclosure.

FIGS. 11A to 11D are diagrams illustrating a user interface providing a 3D human body shape through which a pain area may be specifically input according to an embodiment of the present disclosure.

FIGS. 12A and 12B are diagrams illustrating a screen for outputting pain management information according to surrounding environment information according to an embodiment of the present disclosure.

FIG. 13 is a diagram showing a notification message indicating whether medicine for pain has been taken according to an embodiment of the present disclosure.

FIGS. 14A to 14D are diagrams illustrating a pain management screen for checking pain of a user according to a predetermined period according to an embodiment of the present disclosure.

FIGS. 15A and 15B are diagrams illustrating a screen in which an activity record of a user is accumulated for each activity according to an embodiment of the present disclosure.

FIGS. 16A and 16B are diagrams illustrating a screen for setting user identification information and authority information in order to manage chronic pain according to an embodiment of the present disclosure.

FIG. 17 is a diagram illustrating a case in which a personalized pain management device is a wearable device according to an embodiment of the present disclosure.

FIG. 18 is a diagram illustrating the configuration of a personalized pain management device according to an embodiment of the present disclosure.

FIG. 19 is a diagram illustrating the contents of sharing information about pain between a personalized pain management device and other devices according to an embodiment of the present disclosure.

### MODE(S) FOR CARRYING OUT THE INVENTION

The terms used in the present specification will be briefly described, and the present disclosure will be described in detail.

Although the terms used in the present disclosure are selected from generally known and used terms while considering functions of the present disclosure, they may vary according to intention or customs of those skilled in the art or emergence of new technology. Some of the terms mentioned in the description of the present disclosure may have been selected by the applicant at his or her discretion, and in such cases the detailed meanings thereof will be described in relevant parts of the description herein. Thus, the terms used in this specification should be interpreted based on the substantial meanings of the terms and the whole content of this specification rather than their simple names or meanings.

In the entire specification, when a certain portion “comprises or includes” a certain component, this indicates that the other components are not excluded and may be further included unless specially described otherwise. The terms “unit”, “-or/er” and “module” described in the specification indicate a unit for processing at least one function or operation, which may be implemented by hardware, software or a combination thereof.

Hereinafter, embodiments of the present disclosure will be described in detail with reference to the accompanying drawings so as to be easily carried out by those having ordinary skill in the technical field to which the present disclosure pertains. The present disclosure may, however, be embodied in various different forms and is not limited to the embodiments set forth herein. In addition, in the drawings, in order to clearly describe the present disclosure, parts that are not directly related to the description are omitted. Further, like reference numerals refer to like elements throughout the specification.

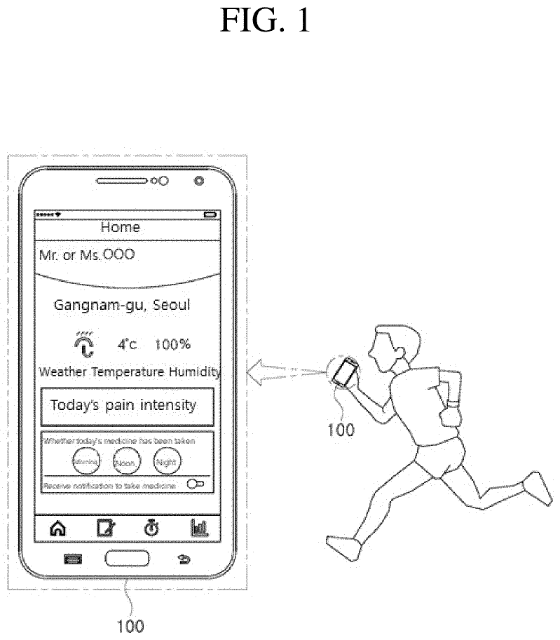

FIG. 1 is a diagram schematically illustrating an operation of a device for providing a personalized pain management method according to an embodiment of the present disclosure.

As illustrated in FIG. 1, a personalized pain management device 100 may provide a method for checking and caring for chronic pain of a user.

According to an embodiment, a user is a person suffering from pain and means any person who may use a personalized pain management device. Hereinafter, expressions such as a user, another user, an individual, and a patient may be used interchangeably, but they have the same meaning in interpretation.

According to an embodiment, the personalized pain management device 100 may be an electronic device such as a mobile phone, a smartphone, a laptop computer, a tablet PC, an electronic-book terminal, a terminal for digital broadcasting, a Personal Digital Assistant (PDA), a Portable Multimedia Player (PMP), a navigation device, an MP3 player, a digital camera, glasses, and a wrist watch, and may include all electronic devices including a display.

According to an embodiment, the personalized pain management device 100 receives detailed information about the degree of pain the user feels from a user, and informs the user of whether the pain is relieved or worsened by the surrounding environment such as weather, air pressure, temperature, and humidity. In addition, the personalized pain management device 100 may obtain information on pain the user feels over time through continuous checking of the pain relief intensity of the user according to whether medicine has been taken.

In addition, by setting a baseline that becomes a threshold point to which the user is able to perform activities without feeling pain, the personalized pain management device 100 may check time or number of times that the user is able to endure the accompanying pain while performing a specific activity. In addition, the personalized pain management device 100 may check the change in pain the user feels by analyzing time or number of times that the user is able to endure the pain, and may provide systematic pain management information by providing information on accumulated pain in a graph.

In addition, the personalized pain management device 100 may obtain pain information of a user clearly and specifically by providing a user interface to input more detailed pain information.

In other words, the personalized pain management device 100 may provide an effect of tracking and managing pain in such a way that pain of a user may be relieved by using information on surrounding environment and user biometric information that cause pain. Hereinafter, the configuration and technical features of the personalized pain management device 100 will be described in more detail.

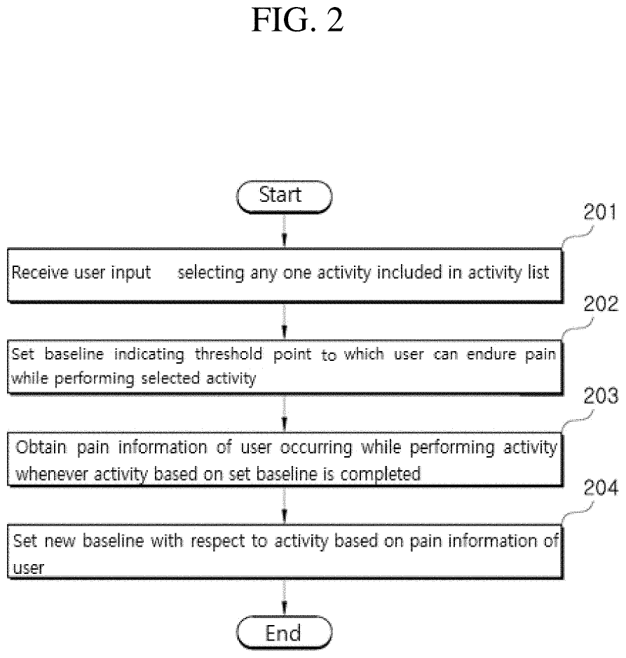

FIG. 2 is a flowchart illustrating a personalized pain management method according to an embodiment of the present disclosure.

According to an embodiment, in block 201, the personalized pain management device 100 may receive user input selecting any one activity from an activity list of a user.

According to an embodiment, the term “activity” refers to an action accompanied by the movement of a user, and means that the user moves in order to achieve a predetermined purpose. In the present disclosure, the activity may cause a pain of a user as the user moves. For example, the activities may include housework, walking, mountain climbing, washing dishes, and hanging up laundry, and may include all actions that cause a user to move or cause a pain.

The activity list of the user may include at least one activity selected from a plurality of preset activities or an activity directly designated by the user.

According to an embodiment, the personalized pain management device 100 may display an activity list including a plurality of activities on the display and receive user input selecting any one activity from the user. The user input according to an embodiment may include an input through a touch pad, a voice input, an input through a keypad, a gesture input, and an input through other devices, and the user input method is not limited.

According to an embodiment, in block 202, the personalized pain management device 100 may use one or more question prompts or user setup request messages so as to set a baseline indicating a threshold point to which the user is able to endure pain while performing the activity selected by the user. According to an embodiment, the threshold point may mean a time or a number of times during which a specific activity may be performed without pain.

According to an embodiment, when a baseline is determined using one or more question prompts, there is an effect that the baseline may be more carefully set by providing and selecting a threshold point to a user step by step. According to an embodiment, when a baseline is determined using a user setup request message, there is an effect that the baseline can be more quickly set by allowing the user to directly set a target value. In addition, there is an effect of increasing the psychological stability of the user by allowing the user to set a target value using one or more question prompts or user setup request messages. Furthermore, in performing pain management according to keeping and increasing the baseline, it is possible to make the user feel a sense of achievement in achieving the goal, thereby helping the psychological pain management effect.

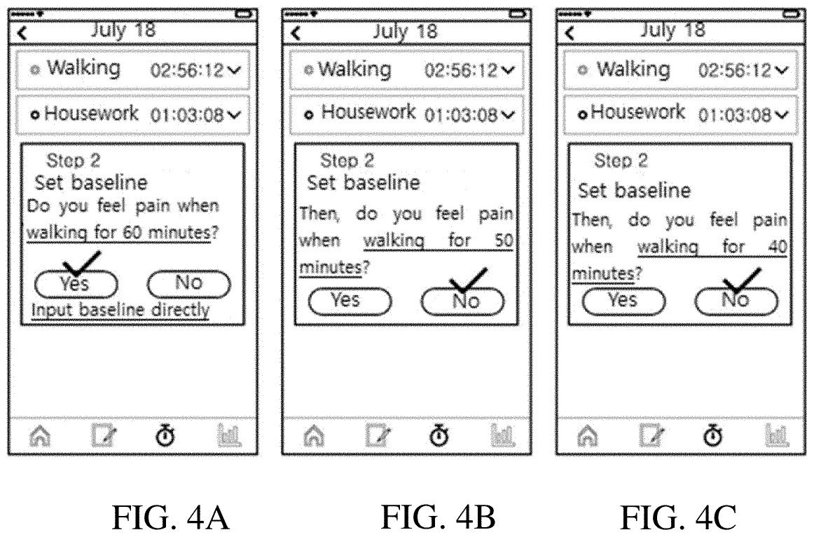

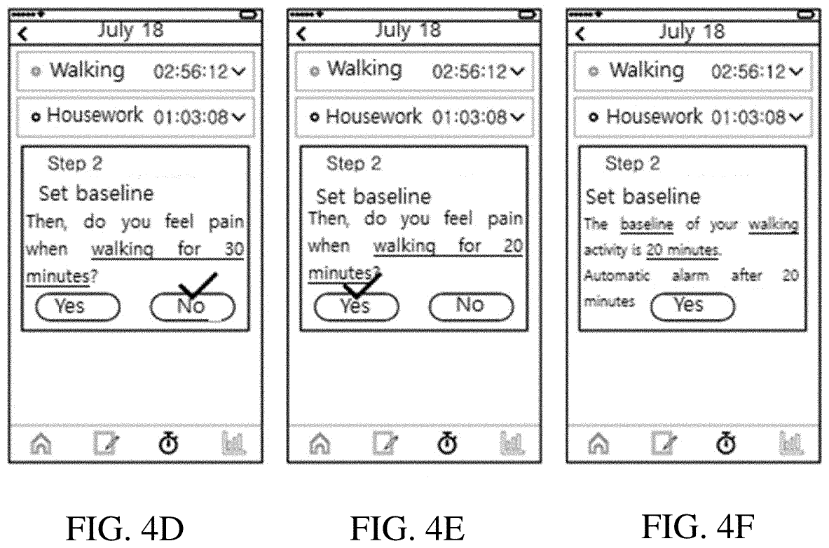

Here, the baseline is an initial set value used to compare the degree of pain after the activity is completed, and is a value set before the activity of a user proceeds. According to an embodiment, the baseline may be set for each activity, and refers to the longest time a user is able to endure without pain when performing a specific activity. Furthermore, the maximum number of activities that a user is able to endure without pain in performing a specific activity may be included. Features of setting the baseline will be described in detail in FIGS. 4A to 4F.

The personalized pain management device 100 may adjust and provide a time to endure pain, and allow the user to select a time to perform the activity without pain, thereby obtaining a personalized baseline for each activity. In addition, in the case of an activity that is checked by the number of times according to another embodiment, the baseline may be obtained using the number of times the user may endure pain.

According to an embodiment, in block 203, the personalized pain management device 100 may obtain pain information of the user occurring while performing the activity from the user whenever an activity based on the set baseline is completed.

For example, when the baseline for the walking activity is 20 minutes, the personalized pain management device 100 may obtain information on the pain change of a user after the walking activity for 20 minutes is completed. The personalized pain management device 100 may obtain information on the pain change of the user directly from the user, or may obtain information from other devices of the user (for example, a wearable watch, and a user terminal).

According to an embodiment, the pain information of the user obtained directly from the user may include at least one information among an activity performance time, a baseline value, a number of steps of a user, whether medicine for pain has been taken, a pain intensity, a pain area, and pain duration. In addition, the pain information of the user obtained from other devices may include sensing information using a device, and may include, for example, a body temperature of a user, a change in skin humidity, and a change in blood flow.

According to an embodiment, in block 204, the personalized pain management device 100 may set a new baseline with respect to the activity based on the pain information of the user.

According to an embodiment, the new baseline may be set immediately after the activity is completed, after the activity has been performed several times, or after a predetermined period of time elapses. According to an embodiment, the new baseline may be set by a user or may be set automatically.

For example, after the activity of the user is completed, the personalized pain management device 100 may obtain information on pain occurring during the activity. For example, the information on whether the pain has occurred within the baseline may be obtained, and when pain has occurred, the information on the intensity and area of the pain may be obtained. The personalized pain management device 100 may store information on the obtained pain.

According to an embodiment, when pain has occurred within the baseline, the personalized pain management device 100 may ask the user whether to reduce and then change the baseline. In addition, when pain has not occurred within the baseline, the personalized pain management device 100 may ask the user whether to expand and then change the baseline.

According to another embodiment, the personalized pain management device 100 may obtain only information on pain and ask whether to change the baseline after a predetermined period of time elapses. For example, after maintaining a baseline of 20 minutes for a walking activity for one week, the device may ask the user whether to set a new baseline. The personalized pain management device 100 may determine and ask whether to reduce or expand the baseline based on the pain information of the user.

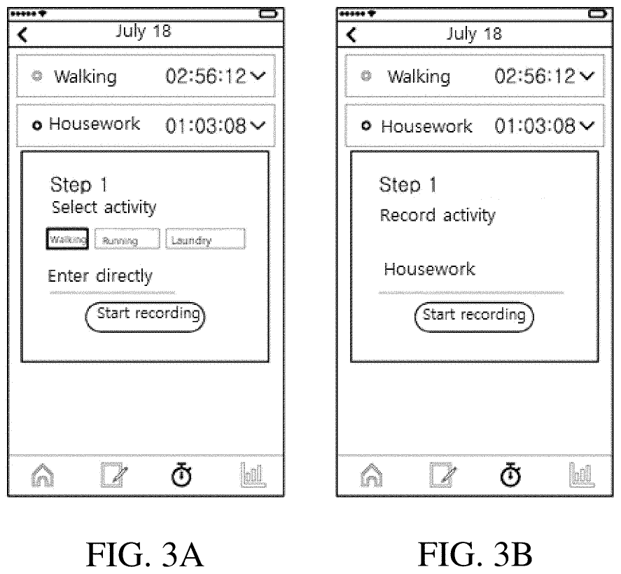

FIGS. 3A and 3B are diagrams illustrating a user interface screen for selecting an activity list according to an embodiment of the present disclosure.

According to an embodiment, the personalized pain management device 100 may provide a user interface through which a specific activity may be selected in order to obtain information on pain occurring while performing a specific activity.

According to an embodiment, FIG. 3A may be an arbitrary activity list provided by the personalized pain management device 100. Alternatively, it may be a list of activities preset by the user. According to an embodiment, the user may select a walking activity from a list of activities such as walking, running, and washing clothes.

In FIG. 3B, the personalized pain management device 100 may receive an activity name directly from a user. Accordingly, a user who wants to manage pain in more detail may manage pain for each activity in detail by specifically designating the activity name in detail. For example, the user may input an activity for doing housework, and the activity input by the user is included in the activity list.

FIGS. 4A to 4F are diagrams illustrating a method of setting a baseline according to an embodiment of the present disclosure.

According to an embodiment, the personalized pain management device 100 may set a baseline for a specific activity by receiving one or more question prompts and user response inputs.

As illustrated in FIG. 4A, the personalized pain management device 100 may display a question prompt as to whether an activity may be performed without pain for a reference period of time. For example, the personalized pain management device 100 may present a question prompt “Do you feel pain when walking for 60 minutes?” When the user sends a response input of “Yes”, it may be determined that 60 minutes are unsuitable to become a baseline for walking activity.

As illustrated in FIG. 4B, when the personalized pain management device 100 receives a response input from the user stating that the activity cannot be performed without pain for a reference period of time, a question prompt as to whether the activity may be performed without pain for a time shortened by a predetermined period of time from the reference period of time may be displayed. For example, the shortened time may be a time shortened by 10% than the previously suggested reference period of time. The ratio of the time to be shortened is not limited to the embodiment and may be designated by the user. According to an exemplary embodiment, the personalized pain management device 100 may shorten a time by 10 minutes than the reference period of time and display a question prompt “Can you perform a walking activity for 50 minutes without pain?”

As illustrated in FIGS. 4C and 4D, the personalized pain management device 100 may ask whether the user is able to walk without pain by continuously shortening the reference period of time.

As illustrated in FIG. 4E, the personalized pain management device 100 may display a question prompt “Can you perform a walking activity for 20 minutes without pain?” and receive a positive response input of “Yes.” The personalized pain management device 100 may determine 20 minutes as the longest time for a user to endure pain among walking activities, and set 20 minutes as a baseline of the walking activity.

As illustrated in FIG. 4F, the personalized pain management device 100 may notify that the baseline of the walking activity is set to 20 minutes.

In addition, according to another embodiment, when a response input indicating that the activity may be performed without pain for a reference period of time is received from the user, a question prompt may be displayed to ask whether the activity may be performed without pain for an extended time than the reference period of time. Next, when the user gives an answer that the user is able to perform the activity without pain for the extended reference period of time, the reference period of time extended once more may be presented. In other words, the personalized pain management device 100 may set a longest time for a user to endure pain during a walking activity as a baseline.

In addition, according to another embodiment, the baseline may be set based on the reference number of times for a specific activity rather than the reference period of time. The method of setting the baseline is the same as in the case in which time is the standard.

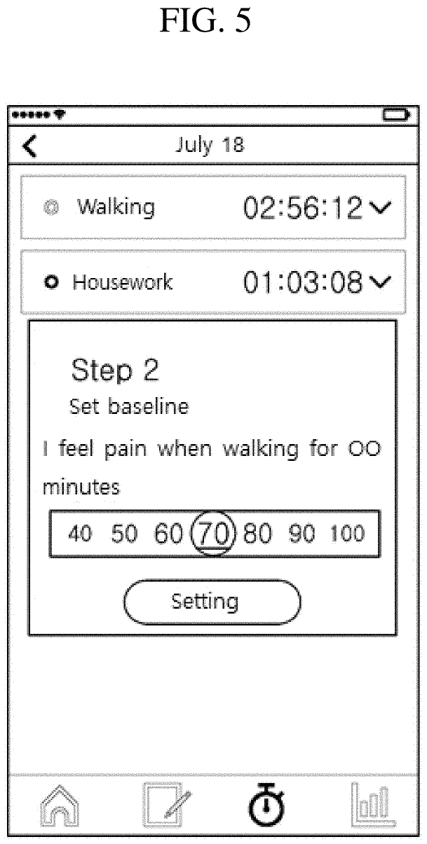

FIG. 5 is a diagram illustrating a method of directly setting a baseline by a user according to an embodiment of the present disclosure.

According to an embodiment, the personalized pain management device 100 may provide a user interface with a user setup request message for directly inputting a baseline for a specific activity. For example, when a baseline for a walking activity is set, a time when a user feels pain when performing a walking activity may be directly set. Accordingly, it is possible to set a baseline for activities more quickly and accurately. In addition, by setting a baseline in which a user may feel a sense of achievement, a method for managing pain having excellent psychological effects may be provided.

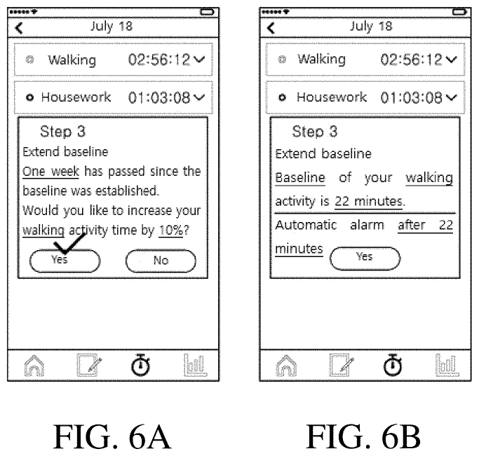

FIGS. 6A and 6B are diagrams illustrating a method of resetting a baseline according to an embodiment of the present disclosure.

According to an embodiment, the personalized pain management device 100 may set a new baseline for an activity. According to an embodiment, the personalized pain management device 100 may automatically change the baseline after a predetermined period has elapsed. Alternatively, the personalized pain management device 100 may ask for content of a user by displaying a message to change the baseline after a predetermined period has elapsed.

As illustrated in FIG. 6A, the baseline may be changed to be reduced or extended based on the existing baseline, and the ratio of the reduction or extension is not limited. For example, it may be set to change by 5-20%, especially 10%, compared to the existing baseline.

Alternatively, the personalized pain management device 100 may determine a baseline change time based on pain information of a user. According to an embodiment, the personalized pain management device 100 may analyze the pain information of a user for one week and determine to reduce the baseline when the frequency, intensity and area of pain during a walking activity within the baseline increase. On the other hand, when it is determined that the pain has decreased through the pain information of a user for one week, the personalized pain management device 100 may make a decision to extend the baseline.

As illustrated in FIG. 6B, when the baseline is changed, the personalized pain management device 100 may display information on the new baseline. In other words, the personalized pain management device 100 can efficiently manage pain of a user by adaptively adjusting the baseline based on pain information over time.

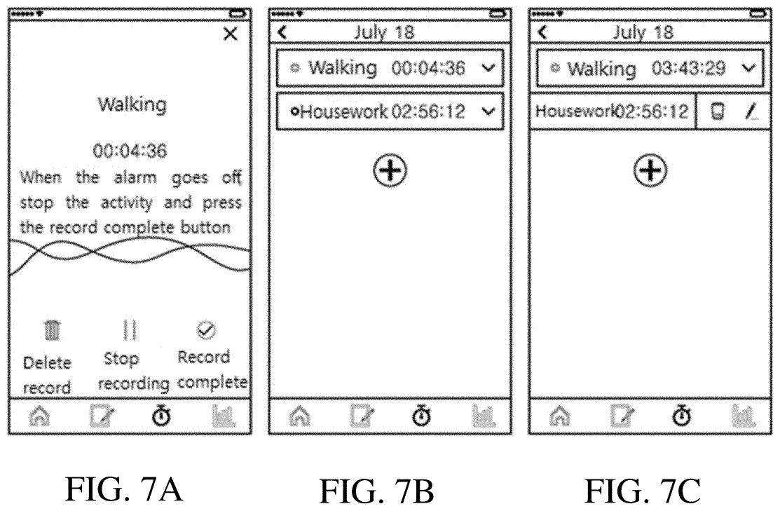

FIGS. 7A to 7C are diagrams illustrating a method of recording an activity of a user according to a baseline according to an embodiment of the present disclosure.

According to an embodiment, when user input selecting an activity for which a baseline is set is received, the personalized pain management device 100 may provide a screen notifying a time when the activity is performed. According to another embodiment, the personalized pain management device 100 may provide a screen notifying the remaining time of the baseline while the activity is being performed.

For example, as illustrated in FIG. 7A, the activity time may be measured at the same time as starting the walking activity. In addition, when the baseline is elapsed while the activity is in progress, it may be notified that the baseline has elapsed. For example, when a walking activity for which a baseline of 20 minutes is set is in progress, a notification may be made at 20 minutes after the user starts the walking activity. In addition, when the baseline means the number of times, the number of activities may be measured while the activity is being performed, and a notification may be made when the baseline has elapsed.

The personalized pain management device 100 may notify that the baseline has elapsed through a visual message, an auditory notification, and a vibration notification.

According to an embodiment, when user input indicating that the activity has been completed is received, the personalized pain management device 100 may obtain the activity time of a user. For example, it may be recorded that the user has performed a walking activity for 4 minutes and 36 seconds.

As illustrated in FIG. 7B, each of a plurality of activities of a user may include an activity record. For example, a walking activity and a housework activity may each have a baseline, and each may have a database for activity time.

According to an embodiment, when user input selecting any one activity is received, an activity record accumulated in each activity may be displayed.

As illustrated in FIG. 7C, the record of a plurality of activities may be deleted or modified. For example, when user input of clicking and dragging any one activity is received, an edit icon and a delete icon may be displayed. According to an embodiment, when the edit icon is selected, the baseline of the activity, the activity name, etc. may be modified, and when the delete icon is selected, the database for the activity may be deleted.

According to an embodiment, a plurality of activities for which a database is set may be continuously performed, or a single activity may be performed alone. For example, a user may perform a plurality of activities continuously, such as washing dishes and then doing laundry, or performing a running exercise while walking, or perform a walking exercise alone. The personalized pain management device 100 may set a break time between a plurality of activities that are continuously performed or after a single activity. A user may balance lives by continuing activities without causing pain through the break time.

When an activity of a user is immoderately performed without a break, since it may adversely affect pain, the personalized pain management device 100 may automatically set a break time in consideration of the baseline criteria. In addition, an appropriate time input by the user may be set as a break time. For example, if the baseline of the walking exercise is 30 minutes and the baseline of the running exercise is 10 minutes, an appropriate break time may be set before the start of the running exercise after the walking exercise is completed, taking into account the health status, age, weight, and medical history of the user.

According to an embodiment, the personalized pain management display 100 may display a screen informing that a break time has started as user input indicating that the walking exercise has been completed is received. For example, if the break time is 5 minutes, a clock for counting 5 minutes and a message indicating the break time may be displayed. The personalized pain management device 100 may display a screen indicating that the running exercise may be started when the break time is completed. In other words, the personalized pain management device 100 may provide an appropriate break time between activities, or after a single activity, and inform the same visually, acoustically, and vibrationally. Therefore, the pain of the user can be effectively managed by allowing the user to take a break consciously so as to manage the body without being hard on the body.

According to an embodiment, when user input notifying that the activity has been completed is not received even after a predetermined period of time has elapsed in excess of the baseline, a pain warning notification may be output. Pain warning notification is a notification notifying previously that pain may occur if the activity continues without stopping.

According to an embodiment, the pain warning notification may include at least one of a visual message, an auditory notification, and a vibration notification.

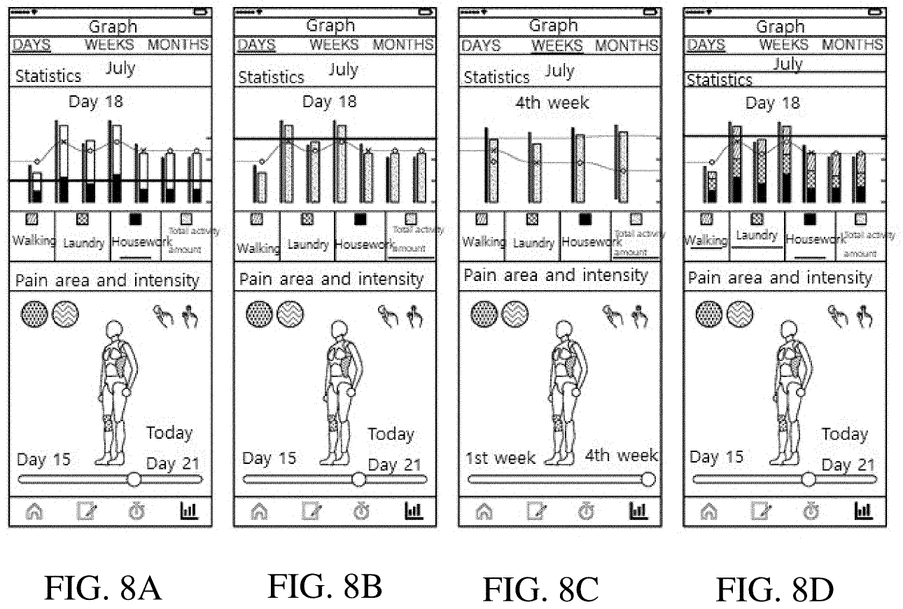

FIGS. 8A to 8D are diagrams illustrating pain information according to activities of a user according to an embodiment of the present disclosure.

According to an embodiment, the personalized pain management device 100 may graphically display pain information using the pain information of a user. According to an embodiment, the pain information of the user may include at least one information among an activity performance time, a baseline value, a number of steps of the user, whether medicine for pain has been taken, a pain intensity, a pain area, pain duration, and surrounding environment information. The type of pain information is not limited.

According to an embodiment, a graph may be generated by an average of how much pain is caused for each pain information, and a pain graph may be created using only pain information selected by the user. Thereby, a user may examine the trend of pain information desired by the user.

According to another embodiment, the personalized pain management device 100 may use a sensor unit including at least one of a temperature sensor that measures a body temperature of the user, a humidity sensor that measures an amount of sweating of the user, a reflective optical sensor that measures a skin characteristic of the user, a heartbeat sensor that measures a heartbeat of the user, a blood pressure sensor that measures a blood pressure of the user, and a motion sensor that measures an activity of the user, and may obtain biometric information of a user. In addition, the personalized pain management device 100 may generate management information on the pain of the user in consideration of at least one of body temperature information, sweating amount information, skin characteristics information, heartbeat information, blood pressure information, and activity information of the user.

According to an embodiment, the personalized pain management device 100 may graphically display the pain information of a user using at least one information and display the pain information in a unit of a predetermined period.

As illustrated in FIG. 8A, the personalized pain management device 100 may provide a pain management graph for a specific activity, respectively. For example, it is possible to provide a graph of each activity time, such as a walking activity, a laundry activity, and a housework activity, a baseline value, whether medicine has been consumed, and an area and intensity of pain. In addition, the personalized pain management device 100 may provide a graph of pain information by date, month, and year.

As illustrated in FIG. 8B, the total amount of activity for a plurality of activities set by the user may be provided as a graph, and the baseline is a sum of the baselines of all activities. In addition, by displaying the total number of steps, it is possible to easily compare and check the amount of activity of the user. The personalized pain management device 100 may show the pain management status by date by showing the total amount of activity every day.

As illustrated in FIG. 8C, the personalized pain management device 100 may show a graph of the total amount of activity for each week. According to an embodiment, the personalized pain management device 100 may clearly indicate the weekly pain management status of the user by displaying an average of a week's worth of accumulated activity amount.

In FIG. 8D, the personalized pain management device 100 may show an activity amount and a pain management status for each individual activity included in the total activity amount. Accordingly, it is possible to check the proportion of each individual activity out of the total amount of activity per day.

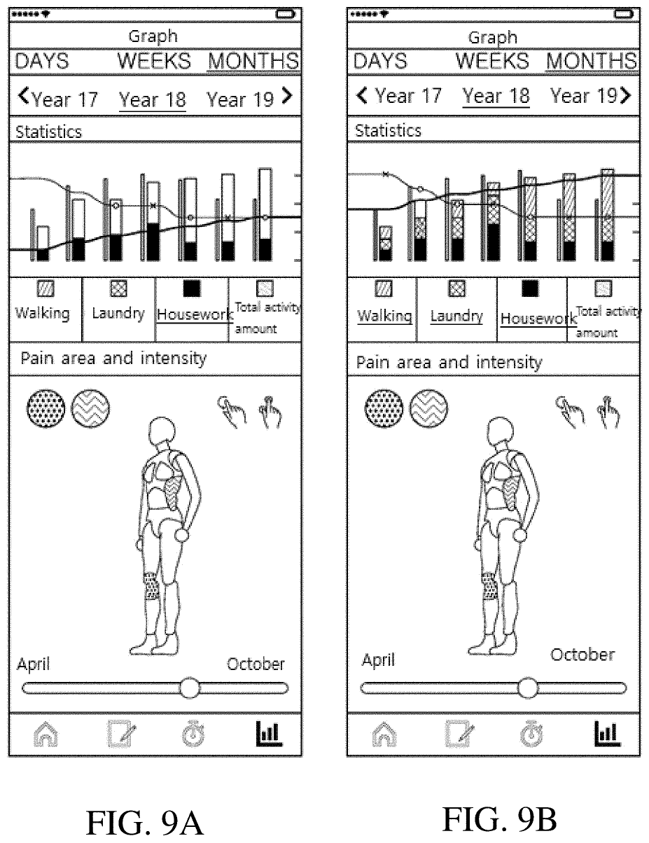

FIGS. 9A and 9B are diagrams illustrating a graph by year in which pain information according to activities of a user is accumulated according to an embodiment of the present disclosure.

Referring to FIG. 9A, the personalized pain management device 100 may provide monthly pain management status. For example, upon reviewing the pain management status of the user in July, it was identified that during housework activities, the user had an average pain intensity of 4, the number of steps was about 60 steps, and the activity time was 30 minutes and 4,000 steps were taken. In comparison with the pain management status in June, the activity time increased according to the increased baseline, and a similar pain intensity was experienced even without taking medicine, indicating that the user's pain was alleviated compared to June.

As illustrated in FIG. 9B, the personalized pain management device 100 shows a graph of the pain management status for total activity. For example, it was identified that the pain intensity of the user has gradually improved on a monthly basis, and the proportion of walking has increased. In conclusion, the personalized pain management device 100 may conclude that the continuous walking activity is helpful in relieving the user's pain, which may be concluded based on the pain-related information of other users.

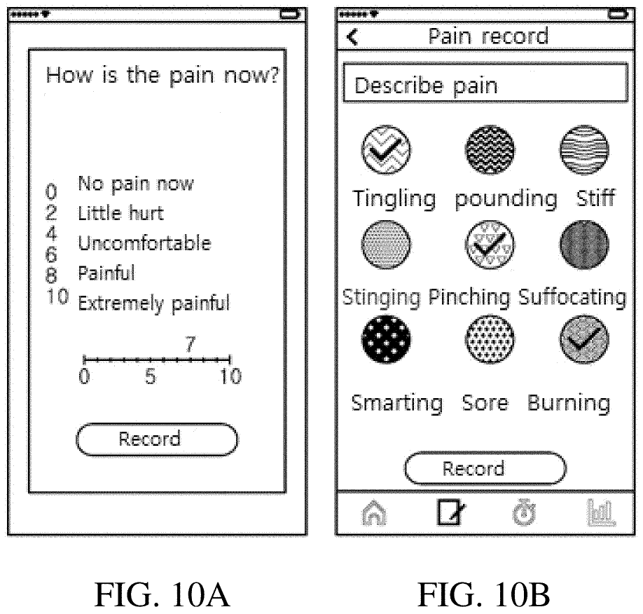

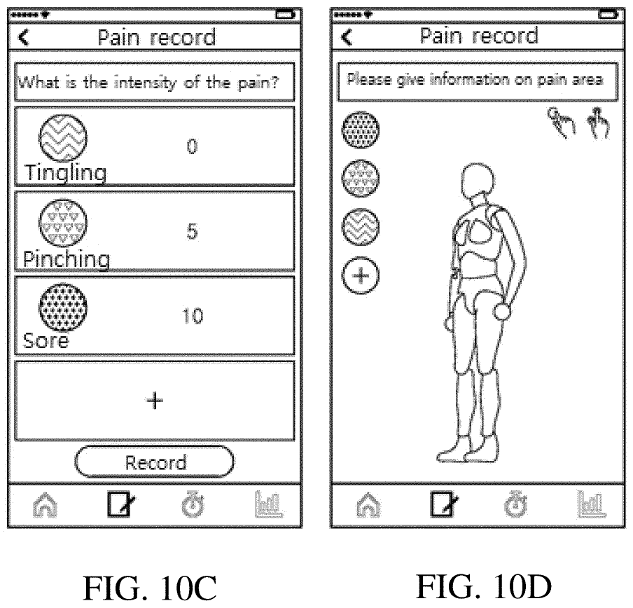

FIGS. 10A to 10D are diagrams illustrating a user interface through which information on pain may be input according to an embodiment of the present disclosure.

According to an embodiment, the personalized pain management device 100 may include an overall intensity of pain, a shape description of pain, an intensity of pain, and an area of pain in order to obtain pain information of the user.

As illustrated in FIG. 10A, the personalized pain management device 100 may provide a user interface for selecting an overall intensity of pain. According to an embodiment, the intensity of pain may be indicated by a number, and as the number increases, it may mean that the pain is severe. For example, when the user selects the intensity of pain of 7, it is highly likely that the user is feeling pain between distress and painfulness.

As illustrated in FIG. 10B, the personalized pain management device 100 may provide a user interface in which a plurality of pain icons visually specifying a feeling related to pain are displayed. The pain icon according to an embodiment is a visual shape of the feeling of pain that a person feels, and for example, a feeling of pain such as a tingling feeling, a pounding feeling, and a pinching feeling are expressed as a visual effect. Characters that describe each visual icon may be accompanied.

According to an embodiment, the user may select a pain icon similar to the feeling of pain the user is experiencing. For example, the personalized pain management device 100 may receive a pain description input of a user who is suffering from a tingling pain, a pinching pain, and a burning pain.

As illustrated in FIG. 10C, the personalized pain management device 100 may provide a user interface for inputting a pain intensity corresponding to at least one selected pain icon. As the intensity of the pain increases, the color of the pain icon may be gradually expressed strongly from yellow to red. In other words, the user may input the degree of pain indicated by the selected pain icon, and the color of the pain icon may be expressed according to the input degree of pain. For example, when a user inputs that a sore pain is felt with an intensity of 10, the icon of a sore pain may be expressed in red. When a user inputs data that a pinching pain is felt with an intensity of 5, the icon of a pinching pain may be expressed in orange.

Next, as illustrated in FIG. 10D, the personalized pain management device 100 may provide a user interface for inputting an area where pain corresponding to at least one pain icon is occurred. The personalized pain management device 100 may receive an area where each pain is felt by providing a character in the shape of a human body.

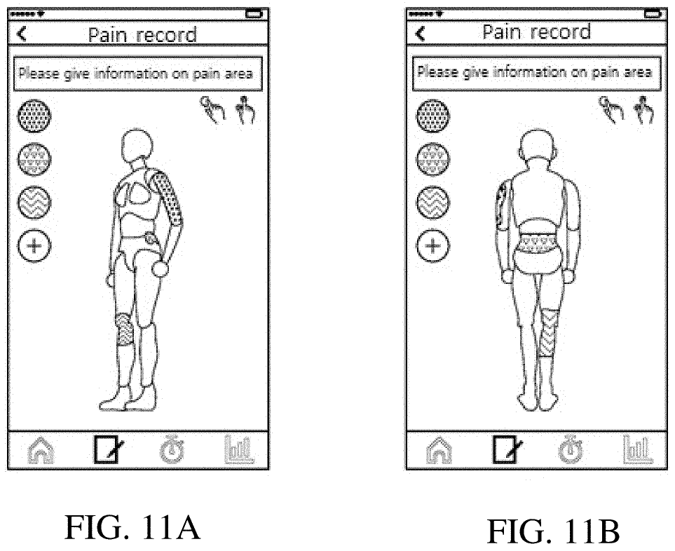

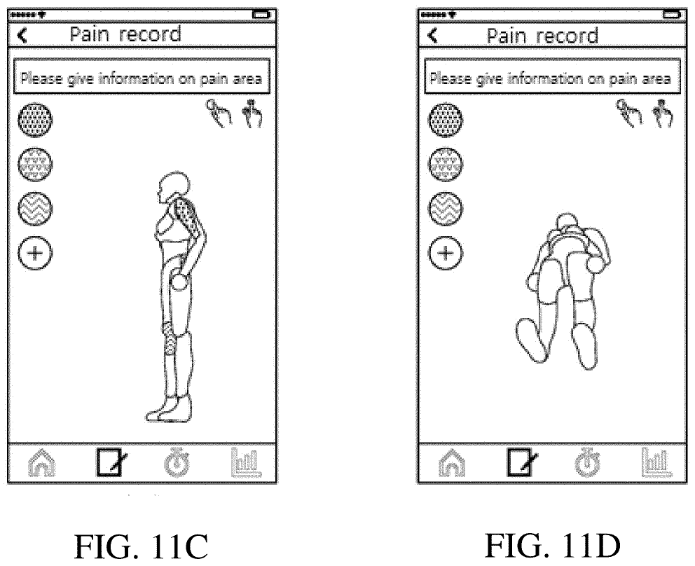

FIGS. 11A to 11D are diagrams illustrating a user interface providing a 3D human body shape through which a pain area may be specifically input according to an embodiment of the present disclosure.

FIGS. 11A to 11D illustrate diagrams providing a shape of a human body in which an area where pain occurs may be input. According to an embodiment, the shape of the human body may be expressed in 3D and may be rotated or enlarged according to an input of a user.

For example, as illustrated in FIGS. 11A to 11D, the shape of the human body viewed from the front, rear side, and lower surface may be provided, and the user selects at least one area of the shapes of the human body to input the area where pain occurs.

According to an embodiment, after receiving a pain icon and a degree of pain from the user, the personalized pain management device 100 may provide a user interface through which the user is able to select an area where pain occurs. For example, when the personalized pain management device 100 receives icons representing “tingling pain” and “pinching pain” and the degree of corresponding pains from the user, a user interface may be provided so that the user may select an area where pain occurs. Thereafter, when a user inputs that a tingling pain has occurred in the right leg, and when a pinching pain has occurred in the lower back, the personalized pain management device 100 may display an icon indicating a tingling pain in the right leg in a color according to the degree of the input pain, for example, yellow, and an icon indicating a pinching pain in the lower back in a color according to the degree of the input pain, for example, orange.

According to an embodiment, the personalized pain management device 100 may provide a user interface for selecting a pain icon and a degree of pain felt in the corresponding area after receiving an input of an area where pain occurs from a user.

Furthermore, the personalized pain management device 100 may provide a user interface for selecting a change in a pain intensity and a pain area according to a change in posture. For example, after the user displays a pain area, the personalized pain management device 100 may change and display a posture of the shape of the human body in which the pain area is displayed. For example, when a user displays that there is pain in the back, a screen may be provided to select whether the pain area is changed or the pain intensity is increased by changing the posture of the shape of the human body to lying, standing, or sitting. The user may select whether the pain area and pain intensity are changed according to the posture through the user interface, and the personalized pain management device 100 may provide an effect of managing the change in pain according to the posture in detail.

Accordingly, the personalized pain management device 100 can more accurately check an area where pain of a user occurs, the feeling of pain, and the intensity of pain, thereby enabling more detailed customized management of pain.

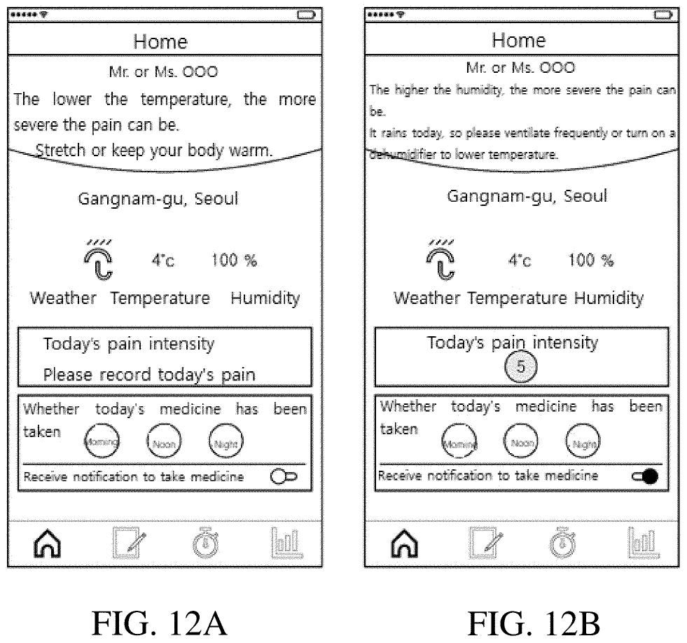

FIGS. 12A and 12B are diagrams illustrating a screen for outputting pain management information according to surrounding environment information according to an embodiment of the present disclosure.

The surrounding environment information according to an embodiment may include at least one of temperature, atmospheric pressure, humidity, time, weather, and fine dust concentration.

The personalized pain management device 100 may generate pain notification information including an effect of surrounding environment information on a pain of a user, and pain relief and prevention information of the user based on the surrounding environment information. In addition, the personalized pain management device 100 may generate pain notification information including the effect of surrounding environment information on the pain of a user, and pain relief and prevention information of the user based on the surrounding environment information, and display pain notification information.

According to an embodiment, the personalized pain management device 100 may receive surrounding environment information from a meteorological administration server, and a weather forecast server, and may measure using sensors included in the personalized pain management device 100.

According to an embodiment, the personalized pain management device 100 may display weather, temperature, and humidity information, and may display pain notification information along with the today's pain intensity of a user and whether today's medicine has been taken.

For example, as illustrated in FIG. 12A, a correlation between temperature and pain of a user may be checked, and it may be determined whether the pain may become more severe according to the current temperature. For example, when the pain may generally be severe as the temperature decreases, the device may indicate that the pain may be severe as the ambient temperature decreases.

In addition, the personalized pain management device 100 may provide pain relief information suitable for a current state of a user. For example, in the case of a weather condition in which pain may worsen, a message to stretch or keep body temperature warm may be displayed to inform the user of information for relieving the pain.

In addition, similarly to FIG. 12B, a message indicating that pain may become severe when the ambient humidity is high may be transmitted. In addition, pain relief information in humid weather may be generated and displayed.

Accordingly, there is an advantage in that the user is able to check pain management information suitable for the weather at once along with his or her pain intensity and whether medicine has been taken.

Furthermore, the personalized pain management device 100 may help the user to take medicine in time by displaying a time when medicine has been taken in the case in which the medicine needs to be taken multiple times a day.

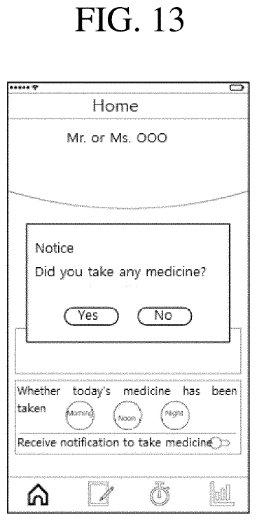

FIG. 13 is a diagram showing a notification message indicating whether medicine for pain has been taken according to an embodiment of the present disclosure.

The personalized pain management device 100 may display a notification message to the user in accordance with a period in which medicine for pain should be taken. For example, the personalized pain management device 100 may display a notification message in the form of a pop-up message every predetermined period of time to take medicine. In addition, the personalized pain management device 100 may notify a timing to take medicine in the form of other notifications such as SMS, social message, and mail. Furthermore, the personalized pain management device 100 may check whether medicine has been taken on the screen when receiving a response from the user that medicine has been taken, and notify a timing to take medicine over again when receiving a response from the user that medicine has not been taken.

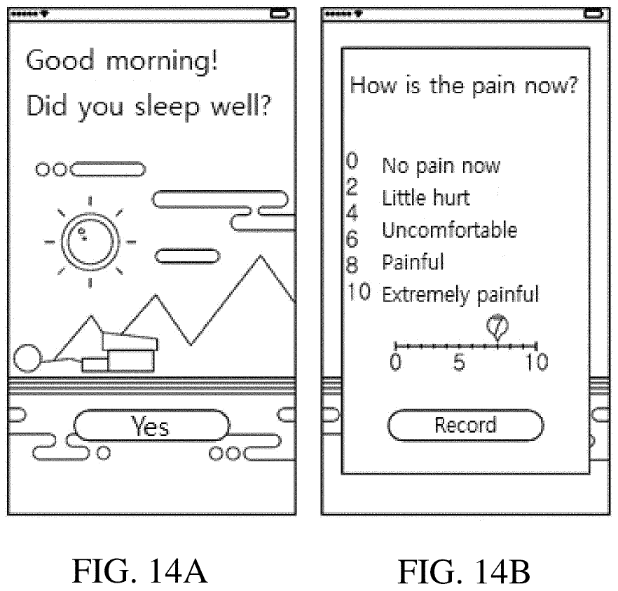

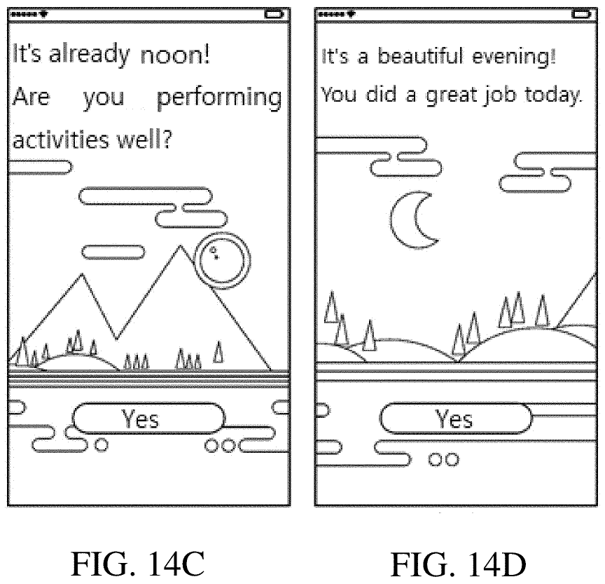

FIGS. 14A to 14D are diagrams illustrating a pain management screen for checking pain of a user according to a predetermined period according to an embodiment of the present disclosure.

The personalized pain management device 100 may provide a user interface for inputting a pain degree according to a preset period. For example, as illustrated in FIGS. 14A, 14C, and 14D, a screen for inputting the degree of pain three times a day in the morning, midday, and evening may be provided.

When the user selects a response input stating that the pain degree is input, the personalized pain management device 100 may provide a user interface screen indicating a current degree of pain of the user in a numerical value. As illustrated in FIG. 14B, the personalized pain management device 100 may store a pain numerical value selected by the user on a screen indicating the degree of pain in a numerical value.

According to an embodiment, the personalized pain management device 100 may generate an average value of a plurality of pain numerical values selected by a user according to a preset period and provide the average value to the user interface screen.

Furthermore, the personalized pain management device 100 may provide information on a pain state of a user by analyzing pain numerical values. In addition, the personalized pain management device 100 may generate accumulated pain numerical value information by converting the pain numerical values into a database.

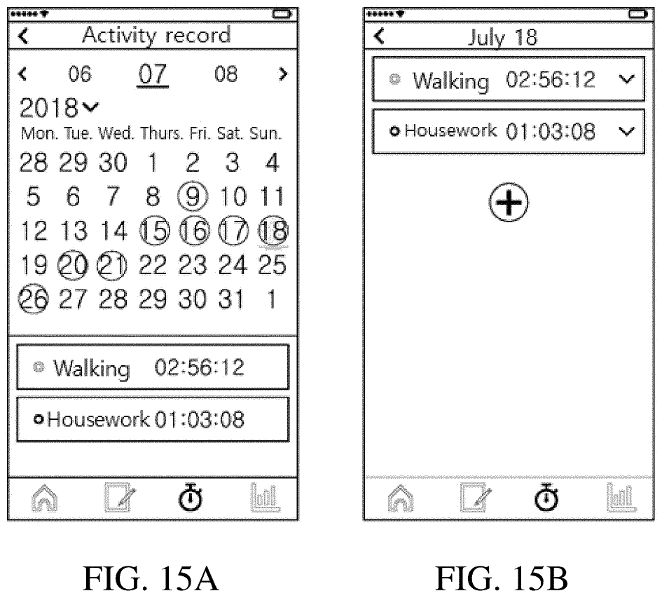

FIGS. 15A and 15B are diagrams illustrating a screen in which an activity record of a user is accumulated for each activity according to an embodiment of the present disclosure.

According to an embodiment, the personalized pain management device 100 may store pain information according to an activity of a user. The pain information may include at least one information among an activity performance time, a baseline value, a number of steps of a user, whether medicine for pain has been taken, a pain intensity, a pain area, pain duration, and surrounding environment information.

The personalized pain management device 100 may store pain information for each day, and may display information about pain on a calendar as illustrated in FIG. 15A. For example, when a user selects July 18, the activity history performed the selected date may be checked.

Furthermore, as illustrated in FIG. 15B, the personalized pain management device 100 may provide a screen in which the activity of July 18 is recorded in detail. Thus, a user may examine an activity and progress of pain by time, and thus may easily manage pain.

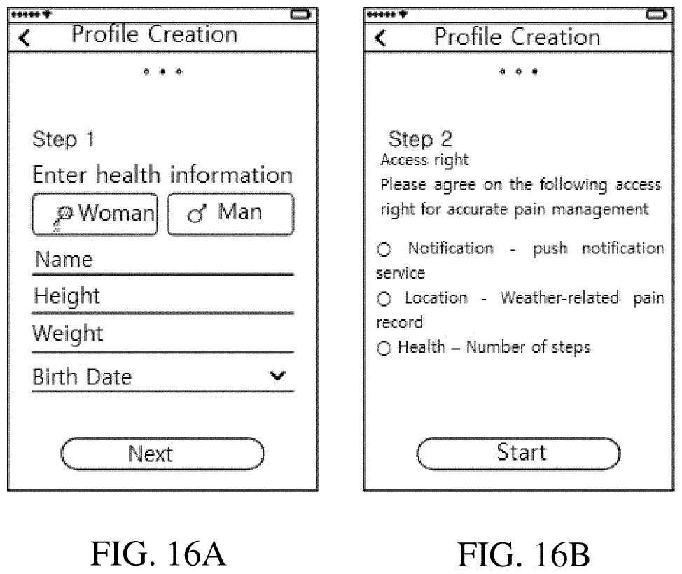

FIGS. 16A and 16B are diagrams illustrating a screen for setting user identification information and authority information in order to manage chronic pain according to an embodiment of the present disclosure.

The personalized pain management device 100 may proceed with membership registration for an initial user. The personalized pain management device 100 may receive user identification information and user authority information in order to efficiently manage pain.

The personalized pain management device 100 may obtain user identification information by providing a screen for inputting personal identification information such as a name, height, weight, and date of birth of a user, as illustrated in FIG. 16A.

Personal information such as a name, height, weight, and date of birth obtained from the user according to an embodiment may be usefully used also when a BMI of the user is calculated in the future. BMI is a measure of the degree of obesity, and represents a value obtained from the calculation of weight (kg) divided by height (m) in meters squared. In general, pain is greatly affected by the weight gain of the user. When the weight of the user increases, the nerves of the user are pressed, and thus, the pain may worsen. Accordingly, by using personal information to manage the BMI of the user, it is possible to provide an effect of managing the pain of the user more accurately and specifically.

In addition, as illustrated in FIG. 16B, the personalized pain management device 100 may check whether to agree on an access right for accurate pain management. For example, the personalized pain management device 100 may obtain an access right to a push notification service, a location service for obtaining surrounding environment information such as weather, and a health-related service capable of obtaining a sleep environment measurement of a user, and a number of steps of a user.

According to an embodiment, the personalized pain management device 100 may provide accurate pain management to a user by using sleep environment information including information on the sleep time of a user, sleep posture, and surrounding environment during sleep. In other words, the quality of sleep has a great influence on the pain of a user, and thus, when the sleep environment information is measured such that a sleep time is short, a sleep posture changes frequently, or noise from the surrounding environment is generated during sleep, the personalized pain management device 100 may determine that the sleep quality of a user is poor, and accordingly provide appropriate pain management to a user. Accordingly, the personalized pain management device 100 may obtain the right to measure the sleep environment of a user, determine a correlation between the sleep environment and pain, and provide a graph.

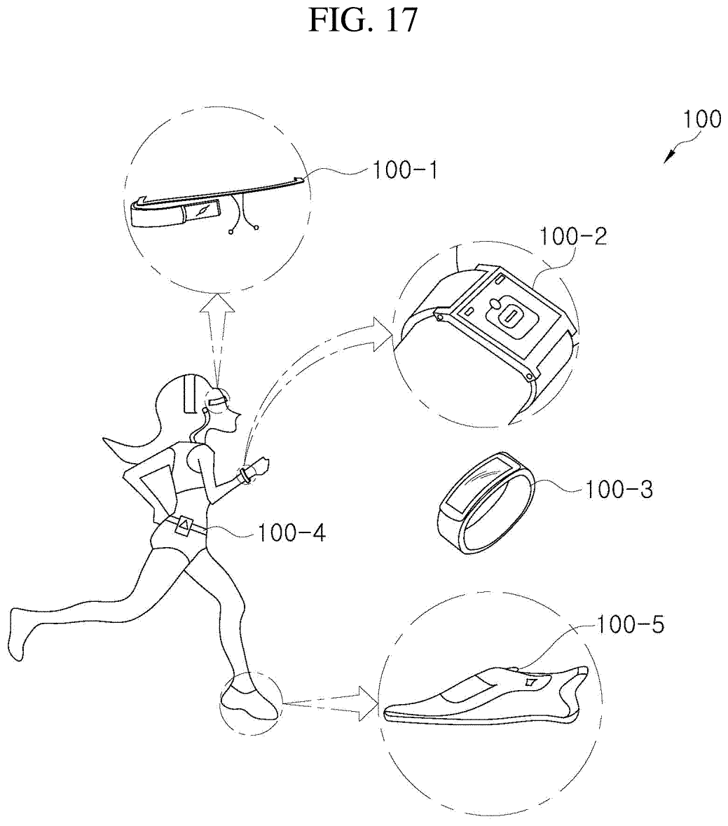

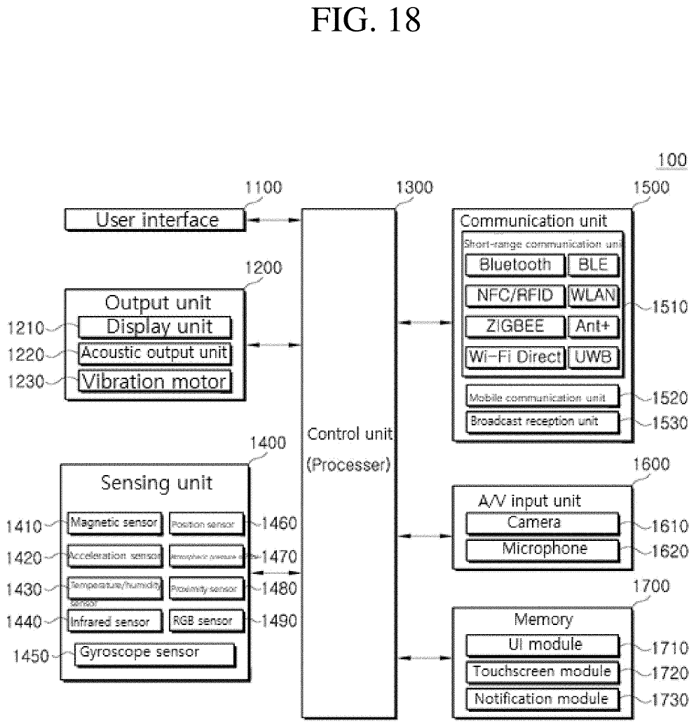

FIG. 17 is a diagram illustrating a case in which a personalized pain management device is a wearable device according to an embodiment of the present disclosure.

According to an embodiment, the personalized pain management device 100 may be a wearable device that may be attached to a body of a user. The personalized pain management device 100 in the form of a wearable device may more accurately sense the movement of a user, so that information related to the pain of a user may be easily obtained.

According to an embodiment, a user may have a plurality of different personalized pain management devices. According to an embodiment, a user may additionally have a plurality of wearable devices 100-1, 100-2, 100-3, 100-4, and 100-5 capable of measuring biometric information.

The plurality of wearable devices 100-1, 100-2, 100-3, 100-4, and 100-5 may directly measure the biometric information of a user. In addition, the personalized pain management device 100 may obtain information on a current state of a user from a plurality of wearable devices 100-1, 100-2, 100-3, 100-4, and 100-5.

The personalized pain management device 100 may efficiently manage the pain information of a user by using the current state information and surrounding environment information of a user obtained from a plurality of wearable devices.

FIG. 18 is a diagram illustrating the configuration of a personalized pain management device according to an embodiment of the present disclosure.

As illustrated in FIG. 18, the personalized pain management device 100 according to an embodiment may include a control unit 1300, a communication unit 1500, and a memory 1700. However, not all of the components illustrated in FIG. 18 are essential components of the personalized pain management device 100. The personalized pain management device 100 may be implemented by more components than the components illustrated in FIG. 18. The personalized pain management device 100 may also be implemented by fewer components than the components illustrated in FIG. 18.

For example, as illustrated in FIG. 18, the personalized pain management device 100 according to an embodiment may further include a user interface 1100, an output unit 1200, a sensing unit 1400, and an A/V input unit 1600 in addition to the control unit 1300, the communication unit 1500, and the memory 1700.

For example, the personalized pain management device 100 according to an embodiment may essentially include the control unit 1300, the user interface 1100, and the memory 1700. The personalized pain management device 100 according to an embodiment may be the personalized pain management device 100 that further includes at least one of the communication unit 1500, the output unit 1200, the sensing unit 1400, and the A/V input unit 1600 in addition to the control unit 1300, the communication unit 1500, and the memory 1700.

The user interface 1100 refers to an element for a user to input data for controlling the personalized pain management device 100. For example, the user interface 1100 includes a key pad, a dome switch, a touch pad (a touch capacitive type, a pressure resistive type, an infrared beam sensing type, a surface acoustic wave type, an integral strain gauge type, a Piezo effect type, or the like), a jog wheel, a jog switch, and the like, but is not limited thereto.

The user interface 1100 may receive user input for controlling an operation of a module installed in the personalized pain management device 100.

According to an embodiment, the user interface 1100 may be included in the personalized pain management device 100 or may exist remotely. According to an embodiment, the user interface 1100 may include at least one open hardware platform supporting the Internet of Things (IoT). For example, a user may transmit user input to the personalized pain management device 100 through at least one open hardware platform in the form of an image, audio, and text.

Furthermore, the personalized pain management device 100 may interlock wirelessly with separate external devices such as speakers, loudspeakers, microphones, cameras, display devices, and drones based on the IoT technology, and may receive user input through the external devices.

The output unit 1200 may output an audio signal, a video signal, or a vibration signal. The output unit 1200 may include a display unit 1210, an acoustic output unit 1220, and a vibration motor 1230. According to an embodiment, the output unit 1200 may output a notification message in the form of an audio, a video, and/or vibration.

The display unit 1210 displays and outputs information processed by the personalized pain management device 100.

The acoustic output unit 1220 outputs audio data received from the communication unit 1500 or stored in the memory 1700. In addition, the acoustic output unit 1220 outputs acoustic signals associated with functions performed by the personalized pain management device 100 (for example, a call signal reception tone, a message reception tone, and a notification sound). For example, the acoustic output unit 1220 may output an alarm sound to notify that an event has occurred.

The control unit 1300 typically controls the overall operation of the personalized pain management device 100. According to one embodiment, the control unit is also referred to as a processor. For example, the control unit 1300 may control the overall operations of the user interface 1100, the output unit 1200, the sensing unit 1400, the communication unit 1500, and the A/V input unit 1600 by executing programs stored in the memory 1700. In addition, the control unit 1300 may perform the functions of the personalized pain management device 100 by executing programs stored in the memory 1700. The control unit 1300 may include at least one processor. The control unit 1300 may include a plurality of processors or may include one integrated processor, depending on functions and roles thereof. According to an embodiment, the control unit 1300 may include at least one processor for providing a notification message by executing at least one program stored in the memory 1700.

The sensing unit 1400 may sense a state of the personalized pain management device 100, a state of a user, or a state around the personalized pain management device 100, and transmit the sensed information to the control unit 1300.

The sensing unit 1400 may include at least one of a magnetic sensor 1410, an acceleration sensor 1420, a temperature/humidity sensor 1430, an infrared sensor 1440, a gyroscope sensor 1450, a position sensor (for example, GPS) 1460, an atmospheric pressure sensor 1470, a proximity sensor 1480, and an RGB sensor 1490, but is not limited thereto. The function of each sensor may be intuitively deduced from the name thereof by those skilled in the art, and thus detailed description thereof will not be provided here.

The communication unit 1500 may include one or more components that enable the personalized pain management device 100 to communicate with another device 100-2 and another server 300. For example, the communication unit 1500 may include a short-range communication unit 1510, a mobile communication unit 1520, and a broadcast reception unit 1530. The other device may be, but is not limited to, a computing device or a sensing device.

According to an embodiment, the server 300 may store all user information recorded in the personalized pain management device 100. According to an embodiment, the server 300 may create a database for pain management information of users. According to an embodiment, the server 300 may provide pain management information to other servers requiring pain management records, such as a hospital server, a pharmaceutical company server, and a public institution server. A user's consent is required in providing the pain management information, and the user's consent may be obtained through an App implemented in the personalized pain management device 100.

According to an embodiment, the pain management information transmitted to another server after obtaining the user's consent may display only the information desired by a viewer, and the implementation method is not limited.

The short-range communication unit 1500 may include a Bluetooth communication unit 1500, a Bluetooth low energy (BLE) communication unit 1500, a near field communication unit 1500, a WLAN (Wi-Fi) communication unit 1500, a Zigbee communication unit 1500, an infrared data association (IrDA) communication unit 1500, a Wi-Fi direct (WFD) communication unit 1500, an ultra wideband (UWB) communication unit 1500, and an Ant+ communication unit, but is not limited thereto.

The mobile communication unit 1500, 1520 transmits and receives a wireless signal to and from at least one of a base station, an external terminal, and a server on a mobile communication network. Here, the wireless signal may include various types of data associated with transmission/reception of a voice call signal, a video call signal, or a text/multimedia message.

The broadcast reception unit 1530 receives a broadcast signal and/or broadcast-related information from the outside through a broadcast channel. The broadcast channel may include a satellite channel and a terrestrial channel. The personalized pain management device 100 may not include the broadcast reception unit 1530 according to some embodiments.

According to an embodiment, the communication unit 1500 may receive a video sequence including a plurality of frames from a camera installed on the personalized pain management device 100. According to an embodiment, the communication unit 1500 may transmit a command for controlling an operation of the module installed on the personalized pain management device 100 to the module installed on the personalized pain management device 100.

The A/V (Audio/Video) input unit 1600 is a unit for inputting an audio signal or a video signal and may include a camera 1610 and a microphone 1620. The camera 1610 may obtain an image frame, such as a still image or a moving picture, through an image sensor in a video call mode or a photographing mode. An image captured through the image sensor may be processed by the control unit 1300 or a separate image processor (not shown). For example, an image captured by the camera 1610 may be utilized as information for determining whether an event has occurred.

The microphone 1620 receives an external acoustic signal and processes the external acoustic signal into electrical voice data. For example, the microphone 1620 may receive an acoustic signal from an external electronic device or a user. The microphone 1620 may use various noise removal algorithms to remove noises generated while an external acoustic signal is being input.

The memory 1700 may store a program for processing and controlling of the control unit 1300 and may store data input to or output from the personalized pain management device 100.

The memory 1700 may include at least one type of storage medium of a flash memory 1700, a hard disk, a multimedia card micro, a card type memory 1700 (for example, an SD memory or an XD memory), a random access memory (RAM), a static random access memory (SRAM), a read-only memory (ROM), an electrically erasable programmable read-only memory (EEPROM), a programmable read-only memory (PROM), a magnetic memory 1700, a magnetic disk, and an optical disc.

Programs stored in the memory 1700 may be classified into a plurality of modules according to their functions. Here, the plurality of modules are software, not hardware, and mean a module that operates functionally, and may be classified into, for example, a UI module 1710, a touchscreen module 1720, and a notification module 1730.

The UI module 1710 may provide a dedicated UI or a dedicated GUI interlocked with the personalized pain management device 100 for each application. The touchscreen module 1720 may sense a touch gesture on a user's touchscreen and transmit information regarding the touch gesture to the control unit 1300. The touchscreen module 1720 according to an embodiment may recognize and analyze a touch code. The touchscreen module 1720 may also be configured as separate hardware including a controller.

The notification module 1730 may generate a signal for notifying an occurrence of an event. The notification module 1730 may output a notification signal in the form of a video signal through the display unit 1210, may output a notification signal in the form of an audio signal through the acoustic output unit 1220, or may output a notification signal in the form of a vibration signal via the vibration motor 1230.

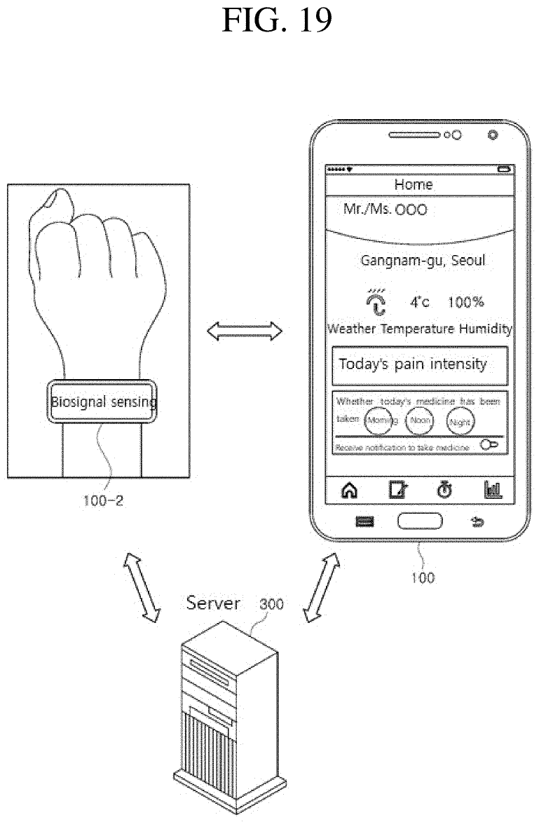

FIG. 19 is a diagram illustrating the contents of sharing information about pain between a personalized pain management device and other devices according to an embodiment of the present disclosure.

According to an embodiment, the personalized pain management device 100 may communicate with other devices 100-2 and servers 300.

According to an embodiment, the other device 100-2 may be a device previously registered by a user or a device of another user. In addition, the other device 100-2 may include a mobile phone, a smartphone, a notebook computer, a digital broadcasting terminal, a personal digital assistant (PDA), a portable multimedia player (PMP), a camera, a navigation device, a tablet computer, an e-book terminal, and a smartwatch. According to an embodiment, when the other device 100-2 is in the form of a wearable device mounted to a user, the personalized pain management device 100 may obtain biosignal information of the user. For example, biosignal information including the pulse rate, body temperature, and skin humidity of a user may be obtained. The personalized pain management device 100 may analyze pain information by using the biosignal information of a user.

In addition, when the other device 100-2 is another user device, the personalized pain management device 100 may share pain information by transmitting information on the pain of a user to another user device. For example, when the other device 100-2 is a device of a family member or a caregiver, there is an advantage in that the family member or the caregiver is able to check the pain state of a user anywhere in real time.

According to an embodiment, the server 300 may include all servers for storing information related to pain, obtaining surrounding environment information, obtaining hospital information, or obtaining information related to pain. The form and type of servers are not limited.

According to an embodiment, the personalized pain management device 100 may transmit and store the pain information of a user to the server 300, and may receive information related to pain from the server 300. For example, pain relief information suitable for a pain state of a user, pain prevention information according to surrounding environment information, and information on pain-related drugs and procedures may be received.

According to an embodiment, the personalized pain management device 100 may recommend at least one of an exercise application that provides an exercise capable of improving pain based on pain information of a user, a medical management application that helps relieve pain, a hospital related to pain, a drug that is effective in relieving pain, and a medical device that helps improve pain.

For example, when user input for selecting any one of a plurality of recommended hospital sites is received, the personalized pain management device 100 may be connected to a hospital server.

In addition, the personalized pain management device 100 may analyze the pain information of a user, generate management information on the pain of a user, and transmit the same to another device 100-1 or the server 300 to receive feedback on the pain of a user.

In other words, the personalized pain management device 100 may obtain information related to the pain of a user using a network and share the pain state of a user so that the pain of a user may be taken care of by an expert or a family member.

The method according to the example embodiments of the present disclosure described above may be implemented through program instructions executable through various components on a computer, and such computer programs may be recorded in computer-readable media. The computer-readable media may include, alone or in combination with the program instructions, data files, data structures, and the like. The program instructions recorded on the media may be those specially designed and constructed for the purposes of the present disclosure or they may be of the kind well known and available to those skilled in the computer software arts. Examples of computer programs may include both machine codes, such as produced by a compiler, and higher-level codes that may be executed by the computer using an interpreter. Examples of the computer-readable recording media include magnetic media such as hard disks, floppy disks, and magnetic tape; optical media such as CD-ROM disks and DVD-ROM disks; magneto-optical media such as floptical disks; and hardware devices that are specially configured to store and execute program instructions, such as ROM, RAM, flash memory, and the like. Examples of computer programs may include both machine codes, such as produced by a compiler, and higher-level codes that may be executed by the computer using an interpreter.

Hereinbefore, although the embodiments of the present disclosure have been described in detail, the scope of right of the present disclosure is not limited to the embodiments and various modifications and improvements thereto made by those skilled in the art using the basic concept of the present disclosure as defined in the accompanying claims will fall within the scope of right of the present disclosure.

1. A method performed by a personalized pain management device, the method comprising:
receiving user input of selecting any one activity included in an activity list;
setting a baseline indicating a threshold point to which a user is able to endure pain while performing the activity selected by the user by using one or more question prompts or user setup request messages;
obtaining pain information of the user occurring while performing the activity whenever an activity based on the set baseline is completed; and
setting a new baseline with respect to the activity based on the pain information of the user, wherein:
the user setup request message is a message requesting that the user directly input a longest time or maximum number of times the user is able to perform the activity without pain; and
the setting of the baseline comprises:
displaying a question prompt as to whether the activity is performed without pain for a reference period of time or reference number of times;
when a response input is received from the user indicating that the activity is performed without pain for the reference period of time or reference number of times, displaying a question prompt as to whether the activity is performed without pain for a period of time or a number of times extended from the reference period of time or reference number of times;
when a response input is received from the user indicating that the activity cannot be performed without pain within the reference period of time or reference number of times, displaying a question prompt as to whether the activity is performed without pain for a period of time or a number of times shorter than the reference period of time or reference number of times; and
setting a maximum period of time or maximum number of times for the user to endure pain as a baseline.

2. The method of claim 1, further comprising: when user input of selecting an activity for which the baseline is set is received,
providing a screen notifying a record of the activity;
notifying that the activity time has passed the baseline;
acquiring the activity time of the user according to user input notifying that the activity has been completed; and
obtaining pain information of the user according to the activity of the user.

3. The method of claim 2, wherein the setting of the new baseline with respect to the activity comprises: after a predetermined period of time has elapsed since the setting of the baseline with respect to the activity, setting a new baseline in which the baseline is extended by 5-20%.

4. The method of claim 2, wherein the setting of the new baseline with respect to the activity comprises: after the activity is completed, displaying a question prompt as to whether the activity has been performed without pain for the baseline; and setting a new baseline based on a response input of the user to the question prompt.

5. The method of claim 2, further comprising outputting a pain warning notification when user input notifying that the activity has been completed is not received even after a predetermined period of time or a predetermined number of times has elapsed in excess of the baseline, wherein the pain warning notification includes at least one of a visual message, an auditory notification, and a vibration notification.

6. The method of claim 2, wherein:
the obtained pain information of the user comprises at least one information among an activity performance time, a baseline value, a number of steps of a user, whether medicine for pain has been taken, a pain intensity, a pain area, and pain duration; and
the pain information of the user is graphically displayed to display the pain information in a unit of a predetermined period by using the at least one information.

7. The method of claim 6, further comprising: generating management information on the pain of the user by analyzing the graphically displayed pain information of the user,
wherein the management information on the pain comprises a change in a pain area and a pain intensity for each activity over time, a pain relief according to whether medicine for pain has been taken, and a pain relief according to a change in the baseline.

8. The method of claim 2, wherein the obtaining of the pain information of the user comprises:
providing a user interface in which a plurality of pain icons visually specifying a feeling related to pain are displayed;
receiving user input of selecting at least one pain icon among the plurality of pain icons;
providing a user interface for inputting a pain intensity corresponding to the at least one selected pain icon;
displaying a shape of a human body in which an area where pain occurs corresponding to the at least one pain icon is input;
changing and displaying a posture of the shape of the human body after an area where pain occurs is received from the user in at least one area of the shape of the human body; and
receiving user input indicating a pain intensity and a pain area in the shape of the human body whose posture has been changed.

9. The method of claim 8, wherein the user interface for inputting the area where pain occurs comprises a shape of a human body expressed in 3D that is rotated or enlarged based on the user input.

10. The method of claim 2, further comprising: obtaining surrounding environment information including at least one of temperature, humidity, atmospheric pressure, time, weather, and fine dust concentration; generating pain notification information including an effect of the surrounding environment information on a pain of a user, and pain relief and prevention information of the user based on the surrounding environment information; and displaying the pain notification information.

11. The method of claim 2, further comprising: providing a user interface screen indicating a current degree of pain of the user in a numerical value according to a preset period; storing a pain numerical value selected by the user; and providing information on a pain state of the user by analyzing the pain numerical value.

12. The method of claim 2, wherein the activity list of the user comprises a plurality of activities added by the user and a plurality of automatically preset activities.

13. The method of claim 2, further comprising:
obtaining biometric information of a user using a sensor unit including at least one of a temperature sensor that measures a body temperature of the user, a humidity sensor that measures an amount of sweating of the user, a reflective optical sensor that measures a skin characteristic of the user, a heartbeat sensor that measures a heartbeat of the user, a blood pressure sensor that measures a blood pressure of the user, and a motion sensor that measures an activity of the user; and
generating management information on the pain of the user in consideration of at least one of body temperature information, sweating amount information, skin characteristics information, heartbeat information, blood pressure information, and activity information of the user.

14. The method of claim 2, further comprising: recommending at least one of an exercise application that provides an exercise capable of improving the pain based on the pain information of the user, a medical management application that helps relieve the pain, a hospital related to the pain, a drug that is effective in relieving the pain, and a medical device that helps improve the pain.

15. The method of claim 2, further comprising:
generating management information on a pain of a user by analyzing the pain information of the user; and
transmitting the information on the pain of the user to an external device,
wherein the external device comprises another terminal registered in advance and a server of a hospital related to pain.

16. A personalized pain management device, comprising:
a user interface capable of receiving user input;
a processor; and
a memory storing instructions executable by the processor,
wherein by executing the instructions, the processor is configured to:
receive user input of selecting any one activity included in an activity list;
set a baseline indicating a threshold point to which a user is able to endure pain while performing the activity selected by the user by using one or more question prompts or user setup request messages;
obtain pain information of the user occurring while performing the activity whenever an activity based on the set baseline is completed; and
set a new baseline with respect to the activity based on the pain information of the user, and
wherein when the baseline is set, the processor is configured to:
display a question prompt as to whether the activity is performed without pain for a reference period of time or reference number of times;
when a response input is received from the user indicating that the activity is performed without pain for the reference period of time or reference number of times, display a question prompt as to whether the activity is performed without pain for a reference period of time or a number of times extended from the reference period of time or reference number of times;
when a response input is received from the user indicating that the activity cannot be performed without pain within the reference period of time or reference number of times, display a question prompt as to whether the activity is performed without pain for a period of time or a number of times shorter than the reference period of time or reference number of times; and
set a maximum period of time or maximum number of times for the user to endure pain as a baseline.

17. The device of claim 16, wherein, when user input of selecting an activity for which the baseline is set is received, the processor is configured to:
provide a screen notifying a record of the activity being performed;
notify that an activity time has passed the baseline;
obtain the activity time of the user according to user input notifying that the activity has been completed; and
obtain pain information of a user according to the activity of the user.

18. The device of claim 17, wherein the processor is configured to:
when a new baseline with respect to the activity is set, display a question prompt as to whether the activity has been performed without pain for the baseline after the activity is completed; and
set a new baseline based on a response input of the user to the question prompt.

19. The device of claim 17, wherein the processor is configured to:
when the pain information of the user is obtained, provide a user interface in which a plurality of pain icons visually specifying a feeling related to pain are displayed;
receive user input of selecting at least one pain icon among the plurality of pain icons;
provide a user interface for inputting a pain intensity corresponding to the at least one selected pain icon;
display a shape of a human body in which an area where pain occurs corresponding to the at least one pain icon is input;
change and display a posture of the shape of the human body after an area where pain occurs is received from the user in at least one area of the shape of the human body; and
receive user input indicating a pain intensity and a pain area in the shape of the human body whose posture has been changed.

20. A computer program stored in a recording medium in order to execute a personalized pain management method in combination with a computer as hardware, the computer program comprising:
receiving user input of selecting any one activity included in an activity list;
setting a baseline indicating a threshold point to which a user is able to endure pain while performing the activity selected by the user by using one or more question prompts or user setup request messages;
obtaining pain information of the user occurring while performing the activity whenever an activity based on the set baseline is completed; and
setting a new baseline with respect to the activity based on the pain information of the user, wherein:
the user setup request message is a message requesting that the user directly input the longest time or maximum number of times the user may perform an activity without pain, and
the setting of the baseline comprises:
displaying a question prompt as to whether the activity may be performed without pain for a reference period of time or reference number of times;
when a response input is received from the user indicating that the activity may be performed without pain for the reference period of time or reference number of times, displaying a question prompt as to whether the activity may be performed without pain for a period of time or a number of times extended from the reference period of time or reference number of times;
when a response input is received from the user indicating that the activity may not be performed without pain within the reference period of time or reference number of times, displaying a question prompt as to whether the activity may be performed without pain for a period of time or a number of times shorter than the reference period of time or reference number of times;
and setting a maximum period of time or maximum number of times for the user to endure pain as a baseline.
